(19) United States
(12) Patent Application Publication
(10) Pub. No.: US 2023/0256056 A1
(43) Pub. Date: Aug. 17, 2023

(54) SUSTAINED RELEASE CANCER THERAPEUTICS FORMULATIONS

(71) Applicant: InSitu Biologics, Inc., Oakdale, MN (US)

(72) Inventors: William J. Taylor, Woodbury, MN (US); Kelsey Pflepsen, Minneapolis, MN (US)

(21) Appl. No.: 18/109,028

(22) Filed: Feb. 13, 2023

Related U.S. Application Data
(60) Provisional application No. 63/309,206, filed on Feb. 11, 2022.

(51) Int. Cl. A61K 38/18; A61K 9/16; A61K 31/65

(52) U.S. Cl. CPC A61K 38/1866; A61K 9/1617; A61K 31/65; A61K 9/1652; A61K 9/0019

(57) ABSTRACT

Disclosed herein is a composition for treating cancer comprising: an aqueous carrier, wherein the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%; and a lipid phase comprising an antitumor agent, the lipid phase dispersed within the aqueous carrier, wherein the lipid phase comprises a plurality of lipid microparticles.

### CROSS-REFERENCE TO RELATED APPLICATION(S)

This application claims the benefit under 35 U.S.C. § 119(e) to U.S. Provisional Application 63/309,206, filed Feb. 11, 2022, and entitled “Sustained Release Cancer Therapeutics Formulations,” which is hereby incorporated herein by reference in its entirety for all purposes.

### BACKGROUND

Multifocal tumors have few surgical treatment options in liver, pancreas, renal and lung cancers. There is a need in the art for compositions and methods that are effective in providing sustained localized delivery of antitumor agents to maximize efficacy and minimize systemic effects.

### BRIEF SUMMARY

Disclosed herein is a composition for treating cancer comprising: an aqueous carrier, wherein the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%; and a lipid phase comprising an antitumor agent, the lipid phase dispersed within the aqueous carrier, wherein the lipid phase comprises a plurality of lipid microparticles.

In certain embodiments, a salt form of the antitumor agent unbound by the plurality of lipid microparticles is dissolved in the aqueous carrier. According to further embodiments, a biologic antitumor agent unbound by the plurality of lipid microparticles is dissolved in the aqueous carrier. In exemplary implementations of these embodiments, the biologic antitumor agent is Bevacizumab.

In certain embodiments, the volumetric ratio between the aqueous carrier and the lipid microparticles is from about 70-80 the aqueous carrier to about 30-20 lipid microparticles.

In certain implementations, the lipid microparticles comprise one or more fatty acids having an even number of carbons. In certain embodiments, the one or more fatty acids comprise less than 50% of the total lipid composition of the lipid microparticle.

In further embodiments, the lipid microparticles comprise one or more fatty acids having an odd number of carbons. In exemplary implementations, the one or more fatty acids are chosen from: stearic acid, oleic acid, myristic acid, caprylic acid, capric acid, lauric acid, palmitic acid, arachidic acid, lignoceric acid, cerotic acid, and mixtures of the forgoing and wherein the melting point of the lipid microparticle is above 37° C. In further implementations, the one or more fatty acids comprise a mixture of steric acid and oleic acid and wherein the ratio of steric acid to oleic acid is about 90:10. In yet further implementations, in the lipid microparticles comprise about 12% myristic acid, about 32% palmitic acid, about 10% stearic acid, and about 10% oleic acid. In still further implementations, the lipid microparticles comprise a mixture of lauric acid and caprylic acid, caproic acid, and/or oleic acid.

According to certain embodiments, the lipid microparticles comprise a paraffin, a triglyceride, and/or a wax. In further embodiments, the lipid microparticles comprise a mixture of carnauba wax and caprylic acid, caproic acid, and/or oleic acid.

According to certain embodiments, the plurality of lipid microparticles comprises a first plurality of lipid microparticles and a second plurality of lipid microparticles and wherein the first plurality of lipid microparticles is solid at about 37° C. and the second plurality of lipid microparticles is liquid at 37° C.

In certain embodiments, the lipid microparticle is not a liposome.

According to certain embodiments, the antitumor agent is selected from anthracyclines, mTOR inhibitors, VEGF-TKI agents, and immune stimulators. In exemplary implementations, the antitumor agent is doxorubicin.

In certain implementations, the plurality of lipid microparticles range from about 5 μm to about 20 μm. In further implementations, the plurality of lipid microparticle are about 5 μm or less.

Further disclosed herein is a method of treating cancer in a subject in need thereof comprising administering to the subject and effective amount of a composition comprising: a hydrogel binding matrix; and a plurality of lipid microparticles dispersed within the hydrogel and comprising one or more antitumor agent. In certain implementations, the composition is administered directly to the tumor site by guided needle, laparoscopically or post surgically after removal of the tumor. In further implementations, the composition is administered by way of injection into a solid tumor by way of a guide needle and wherein tumor targeting is verified via ultrasound imaging. In yet further implementations, the antitumor agent is eluted from the composition over a period of between about 4 and about 7 days.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows elution profile of Formulation A, according to certain embodiments.

FIG. 2 shows elution profile of Formulation B, according to certain embodiments.

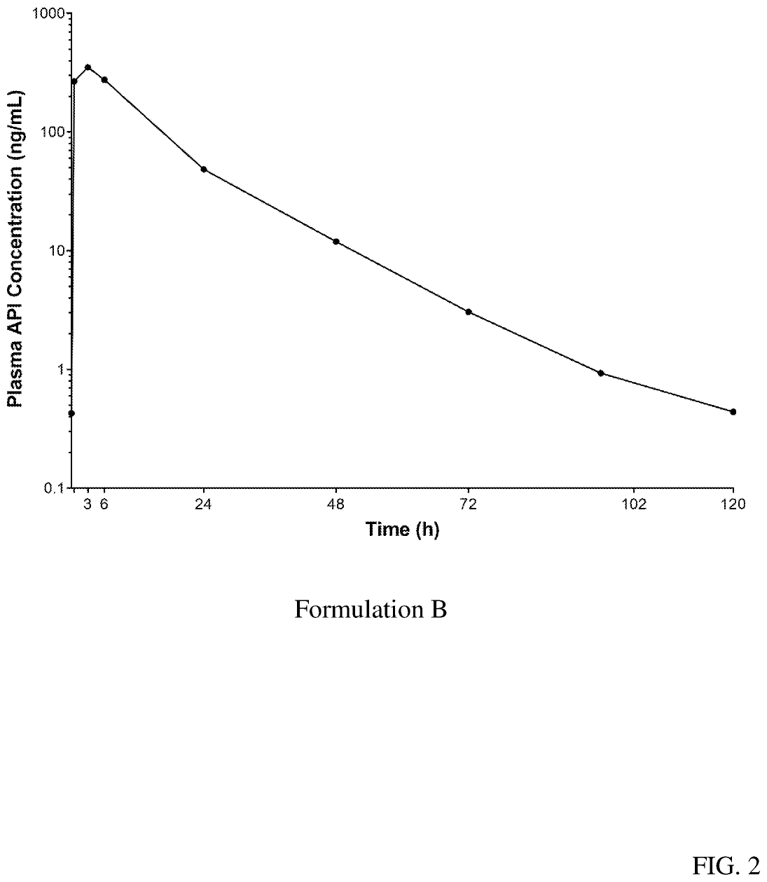

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Perkin Elmer Corporation, U.S.A.).

As used in the specification, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Unless otherwise indicated, references in the specification to parts by weight of a particular element or component in a composition denotes the weight relationship between the element or component and any other elements or components in the composition or article for which a part by weight is expressed, unless expressly described otherwise. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. In some embodiments, the present methods can be used to treat a subject having an epithelial cancer, e.g., a solid tumor of epithelial origin, e.g., lung, breast, ovarian, prostate, renal, pancreatic, or colon cancer.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present invention may be used in combination with MRI or CT, for example, to identify cancer cells.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the identification can be performed by one who subsequently performed the administration.

The terms “antitumor agent” and “chemotherapeutic agent” are used interchangeably herein and refer to an agent for the treatment of cancer. Typically, an antitumor agent is a cytotoxic anti-neoplastic drug, which is administered as part of a standardized regimen. Without being bound by theory, antitumor agents act by killing cells that divide rapidly, one of the main properties of most cancer cells. Preferably, the antitumor agent is not indiscriminately cytotoxic, but rather targets proteins that are abnormally expressed in cancer cells and that are essential for their growth. Non-limiting examples of antitumor agents include: angiogenesis inhibitors, such as angiostatin K1-3, DL-α-Difluoromethyl-ornithine, endostatin, fumagillin, genistein, minocycline, staurosporine, and (±)-thalidomide; DNA intercalator/cross-linkers, such as Bleomycin, Carboplatin, Carmustine, Chlorambucil, Cyclophosphamide, cis-Diammineplatinum(II) dichloride (Cisplatin). Melphalan, Mitoxantrone, and Oxaliplatin; DNA synthesis inhibitors, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; DNA-RNA transcription regulators, such as Actinomycin D, Daunorubicin, Doxorubicin, Homoharringtonine, and Idarubicin; enzyme inhibitors, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A. Tyrphostin AG 34, and Tyrphostin AG 879; gene regulators, such as 5-Aza-2′-deoxycytidine, 5-Azacytidine, Cholecalciferol (Vitamin D3), 4-Hydroxytamoxifen, Melatonin. Mifepristone. Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; microtubule inhibitors, such as Colchicine. Dolastatin 15, Nocodazole, Paclitaxel, Podophyllotoxin, Rhizoxin, Vinblastine, Vincristine, Vindesine, and Vinorelbine (Navelbine); and unclassified antitumor agents, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The antitumor agent may be a neoantigen. Neoantigens are tumor-associated peptides that serve as active pharmaceutical ingredients of vaccine compositions which stimulate antitumor responses and are described in US 2011-0293637, which is incorporated by reference herein in its entirety. The antitumor agent may be a monoclonal antibody such as rituximab, alemtuzumab, Ipilimumab, Bevacizumab, Cetuximab, panitumumab, and trastuzumab, Vemurafenib imatinib mesylate, erlotinib, gefitinib, Vismodegib, 90Y-ibritumomab tiuxetan, 131I-tositumomab, ado-trastuzumab emtansine, lapatinib, pertuzumab, ado-trastuzumab emtansine, regorafenib, sunitinib. Denosumab, sorafenib, pazopanib, axitinib, dasatinib, nilotinib, bosutinib, ofatumumab, obinutuzumab, ibrutinib, idelalisib, crizotinib, erlotinib (Tarceva®), afatinib dimaleate, ceritinib, Tositumomab and 131I-tositumomab, ibritumomab tiuxetan, brentuximab vedotin, bortezomib, siltuximab, trametinib, dabrafenib, pembrolizumab, carfilzomib, Ramucirumab, Cabozantinib, vandetanib, The antitumor agent may be a cytokine such as interferons (INFs), interleukins (ILs), or hematopoietic growth factors. The antitumor agent may be INF-α, IL-2, Aldesleukin, IL-2. Erythropoietin. Granulocyte-macrophage colony-stimulating factor (GM-CSF) or granulocyte colony-stimulating factor. The antitumor agent may be a targeted therapy such as toremifene, fulvestrant, anastrozole, exemestane, letrozole, ziv-aflibercept, Alitretinoin, temsirolimus. Tretinoin, denileukin diftitox, vorinostat, romidepsin, bexarotene, pralatrexate, lenalidomide, belinostat, pomalidomide, Cabazitaxel, enzalutamide, abiraterone acetate, radium 223 chloride, or everolimus. The antitumor agent may be a checkpoint inhibitor such as an inhibitor of the programmed death-1 (PD-1) pathway, for example an anti-PD1 antibody (Nivolumab). The inhibitor may be an anti-cytotoxic T-lyinphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors. DNA methyltransferase inhibitors, histone demethylase inhibitors, or histone methylation inhibitors. The epigenetic drugs may be Azacitidine, Decitabine, Vorinostat, Romidepsin, or Ruxolitinib.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent.

The term “contacting” as used herein refers to bringing a disclosed composition and a cell (e.g., a tumor cell), a target receptor, or other biological entity together in such a manner that the compound can affect the activity of the target, either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, The specific effective amount for any particular subject will depend upon a variety of factors, including the disorder being diagnosed and the severity of the disorder; the specific composition employed; the age, body weight, general health, sex and diet of the patient; the time of administration; the route of administration; the rate of excretion of the specific compound employed; the duration of the diagnosis; drugs used in combination or coincidental with the specific compound employed and like factors well known in the medical arts. For example, it is well within the skill of the art to start doses of a compound at levels lower than those required to achieve the desired diagnostic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. Furthermore, effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein can comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹³N, ¹⁵N, ¹⁸O, ¹⁷O, ³⁵S, ¹⁸F and ³⁶Cl, respectively.

Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes may be used for their ease of preparation and detectability. Further, substitution with heavier isotopes such as deuterium, i.e., ²H, can afford certain advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental Volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules, including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, “drug reservoir” means a phase into which an antitumor agent is dissolved that is dissolved distinct from the carrier phase.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

Disclosed herein are compositions and method for the treatment of cancer. In certain embodiments, the disclosed compositions comprise a hydrogel matrix and plurality of nanoparticles dispersed within the hydrogel and one or more antitumor agent dispersed within the nanoparticles. The disclosed composition and methods allow placement of target materials within desired tissue site in high concentrations that can be eluted from the placed mass at a desired rate. In certain implementations, an antitumor agent can be placed in relatively high concentration in a tumor or tumor site and elute the agent at a desired rate to reduce or eliminate a solid tumor. There are few options for multifocal tumors as found in hepatic, renal and lung cancers. Reducing or eliminating tumors may prolong patient life. The material can be designed to elute the anti-proliferative agent over a several days to ensure the therapeutic agent will be at a locally high enough concentration to make it more effective while reducing systemic concentration of the agent, thereby reducing the symptoms caused by the agent. Many of the anti-proliferative agents work by interfering with DNA replication and cell division. Keeping concentration of these agents locally high will make them more effective. In some cases, the formulation can also be made to be embolic thereby killing the tumor cells directly and then treating the area with a therapeutic agent to ensure all cancer cells have been treated. These are examples of treatments in cancers that would otherwise not have surgical or removal options. In further implementations, liposomes or hydrogel-based drug reservoir particles with endotoxins/pyrogens that elicit a locally large immune response without an active biological agent (i.e. bacteria) to create an infection that may cause tissue damage or if it became systemic, could lead to death. The locally large immune response has promise to activate the patient's immune system to recognize a cancer near the artificially induced infection-like response and attack the tumor cells. IL-2 or similar immune system activators can be used as well to elicit a similar response. Other immunostimulant materials can be supplied in addition to the cancer therapeutic agents either within the same formulation or in addition to the therapeutic agent in a second procedure.

According to certain implementations, the disclosed composition comprises a carrier phase and a drug reservoir phase that contains an API (e.g., an antitumor agent) that is released to a biological system over a targeted treatment duration. In this context the primary function of the carrier phase is to disperse the drug reservoir particles (drug carrying component) to create a stable homogenous mass and allow the use of delivery devices, such as a syringe, to draw up a dose from a container and deliver it to a target tissue, i.e. parenteral injection, intravascular injection, wound instillation, wound packing or mass formation or coating on a tissue surface. In certain embodiments, the drug reservoir is a separate physical phase, a collection of particles, that are contained within the carrier phase but not indistinguishable from the carrier phase. The reservoir phase contains the active pharmaceutical agent dissolved in the reservoir material and may be in an unsaturated, saturated, super saturated, or saturated with pure pharmaceutical phase material (crystals for small molecules) state. In some forms the carrier may also contain the contain the antitumor agent in a different form than the reservoir, such as an antitumor agent salt in an aqueous carrier and the base form antitumor agent in a lipid reservoir. The system is not set to be only aqueous/hydrophobic, but can be opposite, or separate physical phase (polymer).

In certain embodiments, the carrier phase is a hydrogel. The term “hydrogel” as used herein refers to a three-dimensional, hydrophilic or amphiphilic polymeric network capable of taking up large quantities of water. The networks are composed of homopolymers or copolymers (referred to at times herein as a polymer backbone) and are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allow them to swell in aqueous media.

In certain implementations, the hydrogel is comprised of tyramine substituted hyaluronic acid (THA) which is cross linked through di-tyramine linkages. Preparation of THA is described U.S. Pat. No. 6,982,298, which is incorporated herein by reference in its entirety. The degree of tyramine substitution has a significant impact on the properties of the resulting hydrogel. Throughout the instant disclosure, degree of tyramine substitution refers to the percentage of all HA carboxyl groups that have been substituted by tyramine. For example, in a 2% substituted THA, 2% of all HA carboxyl groups have been substituted by tyramine. The percent tyramine substitution within each THA preparation is calculated by measuring: 1) the concentration of tyramine present in the preparation, which is quantitated spectrophotometrically based on the unique UV-absorbance properties of tyramine at 275 nm; and 2) the concentration of total carboxyl groups in the HA preparation, which is quantitated spectrophotometrically by a standard hexuronic acid assay.

As described further below, hydrogel can be tuned to possess a specific osmolality, physical property, antitumor agent elution rate or tissue response by adjusting the concentration of the tyramine substituted polymer backbone, the degree of substitution of the tyramine on the polymer backbone, the molecular weight of polymer backbone, the hydrophilicity of the polymer backbone, the type of polymer backbone and concentration of target molecules, salts, buffers or drug depot (reservoir) particles contained within the hydrogel.

The hydrogel binding matrix consists of a biopolymer backbone containing carboxyl chemical functional groups. Examples of biopolymer backbone include hyaluronic acid and proteins. In certain embodiments, the carboxyl groups are reacted with tyrosine at concentrations from about 0.25% to about 7%. According to certain further embodiments, the tyrosine concentrations is a lower or higher concentration. The examples provided in this disclosure range from about 1 to about 5.5% tyrosine substitution. The tyrosine substituted hyaluronic acid is then placed into solution with the liposome mixture as the solvent phase. In the examples used in this disclosure solutions containing from about 0.1% to about 4% are the preferred concentration. In some cases where the liposomes are substituted for a dense hydrogel particle or mixtures of particles of varying consistency or contain liposomes and dense particles to control delivery rates of target molecules. Dense particles could be 5.5% tyramine substitution and up to and in some cases greater than 10% biopolymer backbone.

Gel density can be used to control rate of elution of an antitumor agent from the gel to the target tissue. A 1% hydrogel will elute antitumor agent for >72 hours, but a 10% gel will extend elution time to over 100 hours. Depending on the antitumor agent or biologic material size and affinity for the hydrogel components, the elution rate can be tuned to a desired elution rate that will allow the hydrogel to act as a drug reservoir for several days.

The hydrogel physical property can also be adjusted by changing the type of polymer backbone. For example, collagen can be used as a polymer backbone, and it is much less hydrophilic than a saccharide-based polymer backbone. The collagen gels do not swell in the same way that polysaccharide gels and have much lower molecular weights and concentrations. It can be envisioned that the polymer backbone can be changed to take advantage of a single polymers physical & chemical characteristics, or several species can be combined in a copolymer or block copolymer in a way that will change the gel physical and chemical properties, the way in which the body interacts with the gel. Some polymers will have a higher affinity rate for an antitumor agent and antitumor agent elution rates will be impacted if a polymer or section of polymer has been chosen that has a higher binding affinity for the antitumor agent. It is also envisioned that by using polymer/antitumor agent combinations in which binding affinity of the antitumor agent to the backbone polymer is pH or temperature dependent, the gel formulation can be adjusted to maximize binding at T=0 and then releasing more antitumor agent as the pH and temperature approaches physiological conditions after exposure to target tissues. In further implementations, antitumor agent diffusion rate is affected by changing the melting point of the lipid microparticles (described further below) as enhanced diffusion can be reached as the liquid-liquid interface (achieved upon melting of the lipid microparticle) diffusion flux is higher than solid to liquid interface.

The hydrogel osmolality can also be tuned by the degree of tyramine substitution, and concentration. Concentrated highly substituted hydrogels by themselves will expel water or undergo syneresis, but by increasing the concentration of the polymer backbone in the example of hyaluronic acid, or adding salts, buffers and/or antitumor agent materials to the formulation the gel can be made to be osmotically neutral or swell slightly. For example, a 5.5% substituted gel can be created that will swell if the backbone polymer concentration is set to 1.5%. It is envisioned that a gel can be created to swell even more as the osmolality of the gel is increased by adding buffers, salts and API ingredients. In certain aspects, the hydrogel is comprised of tyramine substituted hyaluronic acid. According to certain implementations, the hydrogel is formed through di-tyramine crosslinking.

Backbone polymer concentration and degree of substitution may also be tailored to the intended route of administration. Gels with substitution levels 0.1 to 1% allow delivery via needles. 1-2% can be delivered via large diameter needle i.e. 14-16 gauge, and higher than 2% would have to be applied via an applicator at the tumor surface. Higher viscosity gels maybe used to enhance retention at the tumor site.

Hydrogel density can be used to control rate of elution of an antitumor agent from the gel to the target tissue. A 1% hydrogel will elute antitumor agent for >72 hours, but a 10% gel will extend elution time to over 100 hours. Depending on API or biologic material size and affinity for the hydrogel components, the elution rate can be tuned to a desired elution rate that will allow the hydrogel to act as a drug reservoir for several days.

In certain aspects, the degree of tyramine substitution of hyaluronic acid hydroxyl groups ranges from about 0.25% to about 8%. In further aspects, the degree of tyramine substitution of hyaluronic acid hydroxyl groups is about 0.5% to about 3%.

In still further aspects, the tyramine substituted hyaluronic acid is present in the aqueous phase at from about 0.1% to about 4%.

In certain implementations, the tyramine substituted hyaluronic acid is present in the aqueous phase from about 0.1 to about 1%. In further implementations, the tyramine substituted hyaluronic acid is present in the aqueous phase at about 0.25%.

### Lipid Microparticles

According to certain embodiments, lipid microparticles of the disclosed composition are comprised of one or more fatty acids. In certain implementations, the one or more fatty acids have an even number of carbons. In certain implementations, the fatty acids are chosen from: stearic acid, oleic acid, myristic acid, caprylic acid, capric acid, lauric acid, palmitic acid, arachidic acid, lignoceric acid, cerotic acid, and mixtures of the forgoing.

In certain exemplary implementation, where the fatty acid microparticles are comprised of mixtures of fatty acids, the fatty acids are present at specific ratios. For example, in certain implementations, the mixture of fatty acids comprises a 90:10 ratio of steric to oleic acid.

Fatty acids of various carbon lengths are common throughout the living world and are utilized by animals as part of the cell membrane, as energy storage and for thermal regulation. Fatty acids are comprised of carboxylic acid attached to an aliphatic carbon chain. In general, they are insoluble in water but as the carbon chain length shortens, their acidity increases. Fatty acids can be saturated or contain no carbon-carbon double bonds. Or they may be unsaturated, containing one or more carbon-carbon double bonds in the aliphatic carbon chain. Mammalian organisms can process and create fatty acids with even numbered carbon chains. Odd numbered fatty acids are produced by some bacteria and are found in the milk of ruminants, but in most cases, they are even numbered due to the metabolic process that adds two carbons at a time to the chain. Table 1 lists fatty acids typically found in plant and animals. The lipid number lists the number of carbons in the aliphatic chain followed by the number of double bonds. In some listings, the location of the double bond is included with the lipid number. In most cases the fatty acids are usually part of a triglyceride molecule that may contain up to three fatty acids of the same or differing carbon lengths.

In certain implementations, even numbered carbon fatty acids are selected. Mixtures of fatty acids can be made to adjust the melting point of the microparticles. In certain implementations, a mixture of 90% stearic acid with 10% oleic acid is used. This creates a microparticle that melts at 95° F. A similar melting point is achieved by mixing 12% myristic acid 32% palmitic acid, 10% stearic acid, and 10% oleic acid. According to further embodiments, the fatty acid microparticle is formed from a mixture of lauric acid, caprylic acid, and caproic acid. The key factors in choosing a microparticle formulation are melting point and antitumor agent solubility in main component fatty acid. The melting point is important in that particles close to physiological body temperature will be a liquid or soft semi-solid which will increase diffusion rate across a liquid-liquid interface. This may be desirable or not desirable depending on the specific application. In certain embodiments, a combination of low melting point and high melting microparticles (e.g. below and above 37 C) are combined. Antitumor agent solubility will change due to fatty acid chain length and microparticle formulation and it may be desirable to adjust antitumor agent concentration and affinity for the main microparticle fatty acid component. In some formulations increasing molecular weight and chain length of the fatty acid will change solubility of a partially polar antitumor agent counterintuitively. In certain embodiments, the concentration of antitumor agent within a fatty acid microparticle is from about 1-25% by weight.

According to certain alternative embodiments, odd numbered fatty acids are used as an alternative fatty acid in the formulations. Monounsaturated fatty acids such as oleic acid may be used as well alone or in combination with other fatty acids. In certain implementations, poly unsaturated fatty acids can be used, but are not preferable as they oxidize easily and depending on the formulation may polymerize. Monounsaturated fatty acids that are in a cis configuration (most plant sourced) are preferable.

According to certain alternative embodiments, the lipid microparticles comprise one or more triglyceride or a mixture of triglycerides the lipid microparticles comprise one or more triglyceride or a mixture of triglycerides. In further alternative embodiments, the lipid microparticle comprises a paraffin and/or a wax.

TABLE 1
List of Fatty Adds and Corresponding Lipid Numbers.

| | | | Lipid |
|---|---|---|---|
| Common Name | Chemical Name | Structural Formula | Numbers |
| Propionic acid | Propanoic acid | CH₃CH₂COOH | C3:0 |
| Butyric acid | Butanoic acid | CH₃(CH₂)₂COOH | C4:0 |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)₅COOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Carboceric acid | Heptacosanoic acid | CH₃(CH₂)₂₅COOH | C27:0 |
| Montanic acid | Octacosanoic acid | CH₃(CH₂)₂₆COOH | C28:0 |
| Nonacosylic acid | Nonacosanoic acid | CH₃(CH₂)₂₇COOH | C29:0 |
| Melissic acid | Triacontanoic acid | CH₃(CH₂)₂₈COOH | C30:0 |
| Hentriacontylic | Hentriacontanoic | CH₃(CH₂)₂₉COOH | C31:0 |
| acid | acid | | |
| Lacceroic acid | Dotriacontanoic acid | CH₃(CH₂)₃₀COOH | C32:0 |
| Psyllic acid | Tritriacontanoic acid | CH₃(CH₂)₃₁COOH | C33:0 |
| Geddic acid | Tetratriacontanoic | CH₃(CH₂)₃₂COOH | C34:0 |
| | acid | | |
| Ceroplastic acid | Pentatriacontanoic | CH₃(CH₂)₃₃COOH | C35:0 |
| | acid | | |
| Hexatriacontylic | Hexatriacontanoic | CH₃(CH₂)₃₄COOH | C36:0 |
| acid | acid | | |
| Heptatriacontylic | Heptatriacontanoic | CH₃(CH₂)₃₅COOH | C37:0 |
| acid | acid | | |
| Octatriacontylic | Octatriacontanoic | CH₃(CH₂)₃₆COOH | C38:0 |
| acid | acid | | |
| Nonatriacontylic | Nonatriacontanoic | CH₃(CH₂)₃₇COOH | C39:0 |
| acid | acid | | |
| Tetracontylic acid | Tetracontanoic acid | CH₃(CH₂)₃₈COOH | C40:0 |

TABLE 2
Monounsaturated Fatty Acids

| | | | Lipid |
|---|---|---|---|
| | | | Numbers C- |
| Common | | Molecular | Atoms:Double |
| Name | Chemical Name | Formula | Bonds |
| Undecylenic | cis-10-undecenoic acid | C₁₀H₁₉COOH | 11:1 |
| Myristoleic | cis-9-tetradecenoic acid | C₁₃H₂₅COOH | 14:1 |
| Palmitoleic | cis-9-hexadecenoic acid | C₁₅H₂₉COOH | 16:1 |
| Palmitelaidic | trans-9-hexadecenoic | C₁₅H₂₉COOH | 16:1 |
| | acid | | |
| Petroselinic | cis-6-octadecenoic acid | C₁₇H₃₃COOH | 18:1 |
| Oleic | cis-9-octadecenoic acid | C₁₇H₃₃COOH | 18:1 |
| Elaidic | trans-9-octadecenoic acid | C₁₇H₃₃COOH | 18:1 |
| Vaccenic | cis-11-octadecenoic acid | C₁₇H₃₃COOH | 18:1 |
| Gondoleic | cis-9-eicosenoic acid | C₁₉H₃₇COOH | 20:1 |
| Gondolic | cis-11-eicosenoic acid | C₁₉H₃₇COOH | 20:1 |
| Cetoleic | cis-11-docosenoic acid | C₂1H₄1COOH | 22:1 |
| Erucic | cis-13-docosenoic acid | C₂₁H₄₁COOH | 22:1 |
| Nervonic | cis-15-tetracosacnoic | C₂₃H₄₅COOH | 24:1 |
| | acid | | |

In certain embodiments, polyunsaturated fatty acids are used to create the microparticles either alone or in mixtures of other fatty acids. Polyunsaturated fats typically have a lower melting point than do their equivalent carbon number saturated fatty acid analogues. Examples of two essential fatty acids are Linoleic acid (C18:2) and α-Linoleic acid (C18:3). The human body cannot make these fatty acids but requires them and must obtain them through dietary intake. The body can metabolize them so they can be used to generate microparticle drug reservoirs but they have multiple double bonds which oxidize easily and may react with some APIs.

TABLE 3
Omega-3 Fatty Acids

| | | Lipid |
|---|---|---|
| Common name | Chemical name | Numbers |
| Hexadecatrienoic acid | all-cis 7,10,13- | 16:3 (n-3) |
| (HTA) | hexadecatrienoic acid | |
| Alpha-linolenic acid (ALA) | all-cis-9,12,15- | 18:3 (n-3) |
| | octadecatrienoic acid | |
| Stearidonic acid (SDA) | all-cis-6,9,12,15,- | 18:4 (n-3) |
| | octadecatetraenoic acid | |
| Eicosatrienoic acid (ETE) | all-cis-11,14,17- | 20:3 (n-3) |
| | eicosatrienoic acid | |
| Eicosatetraenoic acid (ETA) | all-cis-8,11,14,17- | 20:4 (n-3) |
| | eicosatetraenoic acid | |
| Eicosapentaenoic acid (EPA, | all-cis-5,8,11,14,17- | 20:5 (n-3) |
| Timnodonic acid) | eicosapentaenoic acid | |
| Heneicosapentaenoic | all-cis-6,9,12,15,18- | 21:5 (n-3) |
| acid (HPA) | heneicosapentaenoic acid | |
| Docosapentaenoic acid (DPA, | all-cis-7,10,13,16,19- | 22:5 (n-3) |
| Clupanodonic acid) | docosapentaenoic acid | |
| Docosahexaenoic acid (DHA, | all-cis-4,7,10,13,16,19- | 22:6 (n-3) |
| Cervonic acid) | docosahexaenoic acid | |
| Tetracosapentaenoic acid | all-cis-9,12,15,18,21- | 24:5 (n-3) |
| | tetracosapentaenoic acid | |
| Tetracosahexaenoic | all-cis-6,9,12,15,18,21- | 24:6 (n-3) |
| acid (Nisinic acid) | tetracosahexaenoic acid | |

TABLE 4
Omega 6 Fatty Acids

| | | Lipid |
|---|---|---|
| Common name | Chemical name | Numbers |
| Linoleic acid (LA) | all-cis-9,12- | 18:2 (n-6) |
| | octadecadienoic acid | |
| Gamma-linolenic acid | all-cis-6,9,12- | 18:3 (n-6) |
| (GLA) | octadecatrienoic acid | |
| Eicosadienoic acid | all-cis-11,14- | 20:2 (n-6) |
| | eicosadienoic acid | |
| Dihomo-gamma-linolenic | all-cis-8,11,14- | 20:3 (n-6) |
| acid (DGLA) | eicosatrienoic acid | |
| Arachidonic acid (AA) | all-cis-5,8,11,14- | 20:4 (n-6) |
| | eicosatetraenoic acid | |
| Docosadienoic acid | all-cis-13,16- | 22:2 (n-6) |
| | docosadienoic acid | |
| Adrenic acid (AdA) | all-cis-2,10,13,16- | 22:4 (n-6) |
| | docosatetraenoic acid | |
| Docosapentaenoic acid | all-cis-4,7,10,13,16- | 22:5 (n-6) |
| (Osbond acid) | docosapentaenoic acid | |
| Tetracosatetraenoic acid | all-cis-9,12,15,18- | 24:4 (n-6) |
| | tetracosatetraenoic acid | |
| Tetracosapentaenoic acid | all-cis-6,9,12,15,18- | 24:5 (n-6) |
| | tetracosapentaenoic acid | |

Conjugated fatty acids could also be used alone or in mixtures with other fatty acids to create microparticle drug reservoirs that have desired API solubility/affinity and physical properties.

TABLE 5
Conjugated Fatty Acids

| | | Lipid |
|---|---|---|
| Common name | Chemical name | Number |
| Rumenic acid | 9Z,11E-octadeca-9,11-dienoic acid | 18:2 (n-7) |
| | 10E,12Z-octadeca-10,12-dienoic acid | 18:2 (n-6) |
| α-Calendic acid | 8E,10E,12Z-octadecatrienoic acid | 18:3 (n-6) |
| β-Calendic acid | 8E,10E,12E-octadecatrienoic acid | 18:3 (n-6) |
| Jacaric acid | 8Z,10E,12Z-octadecatrienoic acid | 18:3 (n-6) |
| α-Eleostearic acid | 9Z,11E,13E-octadeca-9,11,13- | 18:3 (n-5) |
| | trienoic acid | |
| β-Eleostearic acid | 9E,11E,13E-octadeca-9,11,13- | 18:3 (n-5) |
| | trienoic acid | |
| Catalpic acid | 9Z,11Z,13E-octadeca-9,11,13- | 18:3 (n-5) |
| | trienoic acid | |
| Punicic acid | 9Z,11E,13Z-octadeca-9,11,13- | 18:3 (n-5) |
| | trienoic acid | |
| Rumelenic acid | 9E,11Z,15E-octadeca- | 18:3 (n-3) |
| | 9,11,15-trienoic acid | |
| α-Parinaric acid | 9E,11Z,13Z,15E-octadeca-9,11,13,15- | 18:4 (n-3) |
| | tetraenoic acid | |
| β-Parinaric acid | all trans-octadeca-9,11,13,15- | 18:4 (n-3) |
| | tetraenoic acid | |
| Bosseopentaenoic | 5Z,8Z,10E,12E,14Z-eicosapentaenoic | 20:5 (n-6) |
| acid | acid | |

The drug reservoir microparticles may also be created from animal ester waxes such as bees wax, vegetable waxes, lanolin and derivatives. Animal ester waxes typically contain triacontanyl palitate and mixtures of palmitate, palmitoleate, oleate esters, triglycerides and aliphatic alcohols. Additives such as cholesterol, triglycerides and aliphatic alcohols may be added to change the physical properties of the microparticles, solubility and affinity of the antitumor agent to the microparticles and act as a carrier molecule to help the antitumor agent diffuse out the microparticle.

Mineral waxes, mineral oils and lanolin derivatives may be added to change physical and chemical properties of the fatty acid microparticle.

Plant sourced waxes can also be used to create the primary phase of the microparticles. Plant waxes provide an advantage over animal waxes in being easier to control environmental conditions and the same organism (palm or plant) lead to lower batch-to-batch variability. Suitable animal and plant waxes are shown in Table 8. In certain embodiments, the fatty acid microparticle is comprised of a carnauba wax. In further embodiments, the fatty acid microparticle is comprised of a combination of carnauba wax and a fatty acid. In exemplary implementations, the mixture is of carnauba wax and oleic acid, caproic acid, caprylic acid, and/or mixtures of the foregoing.

TABLE 6
Examples of Melting points of fatty adds

| Name | Carbon number | Melting point (° C.) |
|---|---|---|
| Capric Acid | 10 | 32 |
| Lauric Acid | 12 | 43 |
| Myristic Acid | 14 | 54 |
| Palmitic Acid | 16 | 62 |
| Stearic Acid | 18 | 69 |
| Arachidic Acid | 20 | 76 |
| Oleic Acid | 18:1 (n-9) | 16 |
| Linoleic Acid | 18:2 | −5 |

TABLE 7
Example of lowering melting temperature
of a stearic acid oleic acid mixture

| OA:SA Ratio | Melting Temp ° C. |
|---|---|
| 0.93 | 32 |
| 0.85 | 37 |
| 0.81 | 45 |
| 0.77 | 42 |
| 0.75 | 47 |
| 0.70 | 51 |
| 0.65 | 48 |
| 0.55 | 57 |
| 0.50 | 56 |
| 0.45 | 59 |
| 0.40 | 60 |
| 0.35 | 63 |
| 0.31 | 64 |

TABLE 2
Source of Common Animal and Plant Waxes

| Name | Source |
|---|---|
| Animal Wax | |
| Beeswax | Insects |
| Lanolin | Sheep |
| Chinese wax | Insects |
| Spermaceti | Sperm Whale |
| Shellac | Insect |
| Plant Wax | |
| Bayberry wax | Bayberry fruit |
| Candelilla wax | Shrubs |
| Carnauba wax | Palm Fronds |
| Castor wax | Castor Bean |
| Esparto wax | Esparto Grass |
| Japan wax | Fruit |
| Jojoba Oil | Seed Simmondsia Chinensis |
| Ouricury wax | Palm Fronds |
| Rice bran wax | Rice Bran |
| Soy wax | Soy Oil |
| Tallow tree wax | Tallow Tree Seeds |

Triglycerides are an alternative to pure fatty acids. They have similar physical properties to the pure counterpart and similar solubility of antitumors. Triglycerides are better tolerated as they are found throughout the body and there are metabolic pathways to absorb and metabolize the lipid. Table 9 lists triglycerides that can be substituted for fatty acids as a lipid drug reservoir particle. In general, even number fatty acid components are selected because the even number fatty acids are more present in tissues. There are some odd number fatty acid triglycerides that are utilized in the body such as triheptanoin found in milk, which are also suitable. Unsaturated fatty acid based triglycerides such as triolein can be used to soften lipid particles or create emulsion droplets if a multiphase formulation is desired. Unsaturated triglycerides are found throughout the body such as tripalmitolein a main component of mammalian fat. Utilizing triglycerides already found in the body increases tolerability and/or reduces likelihood of adverse reactions. In certain embodiments, the concentration of antitumor agent within a triglyceride microparticle is from about 1-16% by weight.

TABLE 3
Triglycerides

| | Fatty Acid | | Fatty Acid |
|---|---|---|---|
| Common Name | Component | Structure | Saturation |
| Tripropionin | Propanoic acid | C₁₂H₂₀O₆ | C3:0 |
| Tributyrin | Butyric acid | C₁₅H₂₆O₆ | C4:0 |
| Trivalerin | Valeric acid | C₁₈H₃₂O₆ | C5:0 |
| Tricaproin | Caproic acid | C₁₅H₂₆O₆ | C6:0 |
| Triheptanoin | Heptanoic acid | C₂₄H₄₄O₆ | C7:0 |
| Tricaprylin | Caprylic acid | C₂₇H₅₀O₆ | C8:0 |
| Tripelarigonin | Pelargonic acid | C₃₀H₅₆O₆ | C9:0 |
| Tricaprin | Decanoic acid | C₃₃H₆₂O₆ | C10:0 |
| Triundcylin | Undecanoic acid | C₃₆H₆₈O₆ | C11:0 |
| Trilaurin | Lauric acid | C₃₉H₇₄O₆ | C12:0 |
| Tritridecanoin | Tridecanoic acid | C₄₂H₈₀O₆ | C13:0 |
| Trimyristin | Myristic acid | C₄₅H₈₆O₆ | C14:0 |
| Tripentadecanoin | Pentadecanoic acid | C₄₈H₉₂O₆ | C15:0 |
| Tripalmitin | Palmitic acid | C₅₁H₉₈O₆ | C16:0 |
| Trimargarin | Margaric acid | C₅₄H₁₀₄O₆ | C17:0 |
| Tristearin | Stearic acid | C₅₇H₁₁₀O₆ | C18:0 |
| Triolein | Oleic acid | C₅₇H₁₀₄O₆ | C18:1, |
| | | | cis 9 |
| Trinonadecanoyl- | Nonadecanoic acid | C₆₀H₁₁₆O₆ | C19:0 |
| glycerol | | | |
| Triarachidin | Arachidic acid | C₆₃H₁₂₂O₆ | C20:0 |
| Triheneicosanoin | Heneicosylic acid | C₆₆H₁₂₈O₆ | C21:0 |
| Trierucin | Erucic Acid | C₆₉H₁₂₈O₆ | C22:cis13, |
| | | | 22:1ω9 |
| Tribehenin | Docosanoic acid | C₆₉H₁₃₄O₆ | C22:0 |
| Tritricosanoin | Tricosanoic acid | C₇₂H₁₄₀O₆ | C23:0 |
| Trilignocerin | Lignoceric acid | C₇₅H₁₄₆O₆ | C24:0 |
| Tripentacosylin | Pentacosylic acid | C₇₈H₁₅₂O₆ | C25:0 |
| Tricerotin | Cerotic acid | C₈₁H₁₅₈O₆ | C26:0 |
| Tricarocerin | Carboceric acid | C₈₄H₁₆₄O₆ | C27:0 |
| Trimontanin | Montanic acid | C₈₇H₁₇₀O₆ | C28:0 |
| Trinonacosylin | Nonacosylic acid | C₉₀H₁₇₆O₆ | C29:0 |
| Trimelissin | Melissic acid | C₉₃H₁₈₂O₆ | C30:0 |
| Trihentriacontylin | Hentriacontylic acid | C₉₆H₁₈₈O₆ | C31:0 |
| Trilacceroin | Lacceroic acid | C₉₉H₁₉₄O₆ | C32:0 |
| Tripsyllin | Psyllic acid | C₁₀₂H₂₀₀O₆ | C33:0 |
| Trigeddin | Geddic acid | C₁₀₅H₂₀₆O₆ | C34:0 |
| Tricerplastin | Ceroplastic acid | C₁₀₈H₂₁₂O₆ | C35:0 |
| Trihexatriacontylin | Hexatriacontylic acid | C₁₁₁H₂₁₈O₆ | C36:0 |
| Triheptatriacontylin | Heptatriacontylic | C₁₁₄H₂₂₄O₆ | C37:0 |
| | acid | | |
| Trioctatriacontylin | Octatriacontylic acid | C₁₁₇H₂₃₀O₆ | C38:0 |
| Trinonatriacontlyin | Nonatriacontylic | C₁₂₀H₂₃₆O₆ | C39:0 |
| | acid | | |
| Tritetracontylin | Tetracontylic acid | C₁₂₂H₂₄₂O₆ | C40:0 |
| Triisopalmitin | Isopalmitic acid | C₅₁H₉₈O₆ | C16:0 |
| Triisostearin | Isostearic acid | C₅₇H₁₁₀O₆ | C18:0 |
| Trilinolein | Linoleic acid | C₅₇H₉₈O₆ | C18:2n-6 |
| Triheptylundecanoin | Heptylundecanoic | C₅₇H₁₁₀O₆ | C18:0 |
| | acid | | |
| Tripalmitolein | Palmitoleic acid | C₅₁H₉₂O₆ | C16:1-8 |
| Triricinolein | Ricinoleic acid | C₅₇H₁₀₄O₉ | C18:1-9, |
| | | | 11-OH |

In certain embodiments, the hydrogel composition contains a plurality of lipid microparticles with varying characteristics in terms of lipid compositions, size, and/or antitumor agent concentration. In these implementations, mixtures of lipid microparticles are used to improve the elution rate of the drug and tune the elution to produce a steady first order release from the particles. Adjusting the particle volume to carrier phase volume ratio will extend the release duration of the antitumor agent.

In exemplary implementations, the fatty acid(s) comprise 50% or less (wt %) of total lipid mass of the lipid microparticles.

In exemplary implementations, the lipid microparticle is not a liposome.

In certain embodiments, the lipid microparticle is formulated so as to be solid upon being implanted into a subject (e.g. at a temperature of about 37° C.) In further embodiments, the lipid microparticle is formulated so as to be a liquid upon being implanted into a subject, with the effect being that elution rate from such liquid microparticles would increases relative to a solid microparticle with a similar concentration of antitumor. In still further embodiments, the composition comprises both of the foregoing microparticles so that some microparticles will remain solid and some will become liquid upon implantation into the subject. The relative balance of the two types of microparticles can be adjusted to achieve the desired elution characteristics.

The size of the lipid microparticle ranges in size from about 1 μm to about 20 μm, in certain implementations. In further embodiments, the lipid microparticle ranges in size from about 5 μm to about 15 μm. In certain exemplary embodiments, the lipid microparticle is about 5 μm. In further exemplary implementations, the lipid microparticle is less than about 5 μm.

In certain implementations, elution properties of the disclosed composition are affected by the volumetric ratio of the aqueous phase to the lipid phase in the composition. According to certain embodiments, the ratio of aqueous to lipid phase is about 50%-80% aqueous phase volume to about 20%-50% lipid phase volume. According to further embodiments, the ratio of aqueous to lipid phase is about 60%-80% aqueous phase volume to about 20%-40% lipid phase volume. According to still further embodiments, the ratio of aqueous to lipid phase is about 70% aqueous phase volume to about 30% lipid phase volume.

According to certain further embodiments, the composition comprises two are more lipid phases within the aqueous carrier phase. In certain implementations of these embodiments, distributed within the aqueous phase is a lipid microparticle phase, as described previously, and a secondary lipid phase which may take the form of an emulsion within the aqueous phase or a plurality of lipid microparticles from which the antitumor agent elutes at a faster rate than the primary lipid microparticle phase. The purpose of the aqueous phase is to carry the microparticles and secondary lipid phase and keep these components homogenous throughout the formulation. It provides volume so that an accurate dose can be delivered to the tumor site and may contain a salt form of the antitumor agent. The salt form of the antitumor agent delivers an upfront burst of drug that matches a similar dose of the saline form of the antitumor agent. The primary lipid phase, or drug reservoir microparticle, contains the largest amount of antitumor agent in base form and will elute the drug component into the aqueous phase slowly after the upfront burst has eluted from the drug product and into the surrounding tissue. There is a mass transfer limitation due to the solubility of the base form in the aqueous carrier phase and the hydrophilic lipophilic balance (HLB) ratio of the microparticles. The base form has a higher affinity for the lipid phase and the lipid phase will always have some antitumor agent present after the elution is complete due to the affinity of the drug for the lipid phase. The secondary lipid phase, or emulsion phase (in some formulations this may be a second type of solid particle), delivers antitumor agent at a faster rate than the solid phase microparticles and together they raise the elution rate in the intermediate phase. Once the targeted duration has been met, the elution rate decreases to zero and is below the pharmaceutically effective dose. In certain embodiments, the composition includes and emulsion phase as described above, but without the plurality of solid microparticles.

Suitable lipids for the secondary lipid emulsion phase are any lipid or mixture of lipids that are liquid at 37°. Examples include, but are not limited to stearic acid, oleic acid, caprylic acid, capric acid, lauric acid, palmitic acid, arachidic acid, lignoceric acid, cerotic acid. In certain embodiments, a mixture of stearic acid and oleic acid are the lipids in the lipid emulsion phase. In further embodiments, triglycerides (e.g. trioleate or tripalmitin and trioleate) form the secondary lipid emulsion phase. According to certain embodiments, an emulsifier is used to stabilize the emulsion. Emulsifiers such as TWEEN or other emulsifiers known in the art are suitable.

In certain implementations, antitumor elution properties of the disclosed composition are affected by the volumetric ratio the two or more lipid phases. According to certain embodiments, the ratio of solid microparticle lipid phase to the emulsion lipid phase is about 50%-75% solid phase volume to about 25%-50% emulsion phase volume. According to certain embodiments, the ratio of solid microparticle lipid phase to the emulsion lipid phase is about 66% solid phase volume to about 34% emulsion phase volume.

### Methods of Formulating Lipid Microparticle Hydrogel Composition

According to certain embodiments, lipid microparticles are generated by agitating a solution of fatty acid phase containing antitumor agent in a much larger volume aqueous phase. The preferred ratio of aqueous to lipid phase is 95%-99.5% aqueous phase to 0.5%-5% lipid phase. It is preferred that the aqueous phase be saturated with the API that is present in the lipid phase. In certain embodiments, a salt in >25 mmol concentration is present in the aqueous phase preferably between 25 and 150 mmol, more preferred to be between 45 and 65 mmol. Tyramine substituted hyaluronic acid is present in the aqueous phase at 0.1% to 4% preferably between 0.1 to 1% and specifically at 0.5% concentration. The two-phase mixture is agitated and cooled until microparticles are generated. The particles are concentrated using a centrifuge, filter or settling tank and the aqueous phase decanted leaving the microparticles behind. Additional aqueous phase containing tyramine substituted hyaluronic acid and horse radish peroxidase is added to the free microparticles and the particles are suspended in the solution at a volume ratio of 30% lipid phase to 70% aqueous phase. A hydrogel is formed with the addition of hydrogen peroxide. The hydrogel maintains particle separation and allows for easy delivery via syringe.

According to certain embodiments, formulations with two or more lipid phases (e.g. lipid microparticle and emulsion) the formulation can be prepared as in the preceding paragraph except that prior to the addition of microparticles to the aquas phase, antitumor agent dissolved in a liquid lipid phase (in certain embodiments a mixture of stearic acid and oleic acid) and mixed vigorously with the aqueous phase until an emulsion is formed. Following the formation of the emulation, the lipid microparticles are added as described previously.

Without wishing to bound to theory, it is believed that the zeta potential is increased by adding the salt (e.g., NaCl) to the aqueous phase, causing the surface charge to increase and cause the particles to repel each other allowing smaller diameter particles to form and preventing coalescing particles from forming larger particles prior to solidification. In certain implementations, the hydrogel comprises between 10 mM and about 70 mM salt. In further implementations, salt concentration is between about 25 mM and about 50 mM salt. In further implementations, the hydrogel comprises at least about 50 mM salt. In certain aspects, the salt is NaCl. As will be appreciated by those skilled in the art, other salts are possible.

In certain implementations of the disclosed composition, the antitumor agent comprises ropivacaine. In exemplary aspects, the ropivacaine is present in the lipid microparticles in an amount of from about 1 to about 25%. In further embodiments, where the lipid microparticles are comprised of triglycerides,

According to certain alternative embodiments, antitumor unbound by the plurality of lipid microparticles is dispersed throughout the hydrogel. According to these embodiments, the API dispersed throughout the hydrogel provides for an immediate burst dose, while the API bound in the lipid microparticles provides for extended sustained release.

In certain implementations, the composition further comprises a radiopaque contrast agent.

Further disclosed herein is a method of treating cancer in a subject in need thereof comprising administering to the subject and effective amount of a composition comprising an immiscible carrier phase and a plurality of lipid microparticles dispersed within the immiscible carrier phase comprising an antitumor agent. In certain implementations, the immiscible carrier phase is a hydrogel, a viscous liquid, a stable emulsion, or a cream.

In exemplary implementations, the immiscible carrier phase is a hydrogel (e.g., a hydrogel comprised of tyramine substituted hyaluronic acid).

In certain embodiments, the antitumor agent is selected from one or more of: angiogenesis inhibitors, such as angiostatin K1-3, DL-α-Difluoromethyl-omithine, endostatin, fumagillin, genistein, minocycline, staurosporine, and (±)-thalidomide; DNA intercalator/cross-linkers, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin: DNA synthesis inhibitors, such as (±)-Amethopterin (Methotrexate), 3-Amino-1,2,4-benzotriazine 1,4-dioxide. Aminopterin, Cytosine β-D-arabinofuranoside, 5-Fluoro-5′-deoxyuridine, 5-Fluorouracil, Ganciclovir, Hydroxyurea, and Mitomycin C: DNA-RNA transcription regulators, such as Actinomycin D. Daunorubicin. Doxorubicin, Homoharringtonine, and Idarubicin; enzyme inhibitors, such as S(+)-Camptothecin, Curcumin, (−)-Deguelin, 5,6-Dichlorobenzimidazole 1-β-D-ribofuranoside, Etoposide, Formestane. Fostriecin, Hispidin, 2-Imino-1-imidazoli-dincacetic acid (Cyclocreatine), Mevinolin, Trichostatin A, Tyrphostin AG 34, and Tyrphostin AG 879; gene regulators, such as 5-Aza-2′-deoxycytidine, 5-Azacytidine, Cholecalciferol (Vitamin D3), 4-Hydroxytamoxifen, Melatonin, Mifepristone. Raloxifene, all trans-Retinal (Vitamin A aldehyde), Retinoic acid, all trans (Vitamin A acid), 9-cis-Retinoic Acid, 13-cis-Retinoic acid. Retinol (Vitamin A), Tamoxifen, and Troglitazone; microtubule inhibitors, such as Colchicine, Dolastatin 15, Nocodazole, Paclitaxel, Podophyllotoxin, Rhizoxin, Vinblastine. Vincristine, Vindesine. and Vinorelbine (Navelbine); and unclassified antitumor agents, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The antitumor agent may be a neoantigen. Neoantigens are tumor-associated peptides that serve as active pharmaceutical ingredients of vaccine compositions which stimulate antitumor responses and are described in US 2011-0293637, which is incorporated by reference herein in its entirety. The antitumor agent may be a monoclonal antibody such as rituximab, alemtuzumab, Ipilimumab, Bevacizumab. Cetuximab, panitumumab, and trastuzumab, Vemurafenib imatinib mesylate, erlotinib, gefitinib, Vismodegib, 90Y-ibritumomab tiuxetan, 131I-tositumomab, ado-trastuzumab emtansine, lapatinib, pertuzumab, ado-trastuzumab emtansine, regorafenib, sunitinib, Denosumab, sorafenib, pazopanib, axitinib, dasatinib, nilotinib, bosutinib, ofatumumab, obinutuzumab, ibrutinib, idelalisib, crizotinib, erlotinib (Tarceva®), afatinib dimaleate, ceritinib, Tositumomab and 131I-tositumomab, ibritumomab tiuxetan, brentuximab vedotin, bortezomib, siltuximab, trametinib, dabrafenib, pembrolizumab, carfilzomib. Ramucirumab, Cabozantinib, vandetanib. The antitumor agent may be a cytokine such as interferons (INFs), interleukins (ILs), or hematopoietic growth factors. The antitumor agent may be INF-α, IL-2, Aldesleukin, IL-2. Erythropoietin, Granulocyte-macrophage colony-stimulating factor (GM-CSF) or granulocyte colony-stimulating factor. The antitumor agent may be a targeted therapy such as toremifene, fulvestrant, anastrozole, exemestane, letrozole, ziv-aflibercept. Alitretinoin, temsirolimus, Tretinoin, denileukin diftitox, vorinostat, romidepsin, bexarotene, pralatrexate, lenalidomide, belinostat, pomalidomide, Cabazitaxel, enzalutamide, abiraterone acetate, radium 223 chloride, or everolimus. The antitumor agent may be a checkpoint inhibitor such as an inhibitor of the programmed death-1 (PD-1) pathway, for example an anti-PD1 antibody (Nivolumab). The inhibitor may be an anti-cytotoxic T-lymphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine, Decitabine, Vorinostat, Romidepsin, or Ruxolitinib

In certain implementations of the disclosed method, the composition is administered to the subject and is delivered near a never or nerve bundle of a subject. In exemplary embodiments, the nerve or nerve bundle innervates the surgical incision area of the subject. The composition may be delivered by way of a syringe or hypodermic needle, other delivery methods known in the art. In exemplary implementations of the disclosed method, the administration of the composition as described herein provides sustained elution of the antitumor agent proximal to the tumor for 110 hours or more.

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

According to certain embodiments, a formulation of anthracycline (doxorubicin) is delivered to a tumor or the space around a tumor via guided needle, laparoscopically or instilled post surgically after removal of the tumor. 100-120 mg of doxorubicin hydrochloride is supplied in hydrogel particles contained within a lipid carrier. In another formulation the doxorubicin base is contained within a lipid drug reservoir nanoparticle carried in an aqueous hydrogel solution.

mTOR

A formulation effective in treating tumors such as renal tumors contain lipid based microparticles containing up to 20 mg of mTOR inhibitor such as Rapamycin (Sirolimus) in a hydrogel carrier. The hydrogel carrier suspends and prevents aggregation of particles prior to injection into the tumor or peritumor site. The lipid component drug reservoir may contain rapamycin in concentrations from 2-20 mg, or higher depending on the lipid component mixture and elution rate. The lipid nanoparticles may be present in volume/volume concentrations between 5% and 50%. The carrier concentration of hydrogel polymer may be between 0.25 and 5.5%. One trained in the art can develop similar formulations for mTOR inhibitors such as Everolimus, Temsirolimus and similar agents.

### VEGF-TKI

Vascular endothelial growth factor (VEGF) and Tyrosine kinase inhibitors (TKI) such as Sorafenib can be presented in lipid nanoparticle drug reservoirs and carried in a hydrogel aqueous formulation and delivered via ultrasound guided needle, fluoroscopy, laparoscopic or visually instilled during surgery to a tumor or peritumor site to deliver a sustained dose of Sorafenib for several days or longer. In one formulation 600-800 mg Sorafenib is contained within lipid nanoparticles in a hydrogel carrier and injected into a solid tumor, injected into the space around the tumor or instilled into a surgical site post tumor excision. Depending on the lipid nanoparticle mixture, a higher load of Sorafenib may be contained within the lipid particles. The lipid nanoparticles may be present in volume/volume concentrations between 5% and 50%. The carrier concentration of hydrogel polymer may be between 0.25 and 5.5%. One trained in the art can develop similar formulations for VEGF-TKI agents such as lenvatinib, axitinib and similar agents.

### Targeted Therapy

Many biologics such as Bevacizumab and interferon have good water solubility and the formulation can be modified to make the hydrogel component the drug reservoir carried within either a water/saline carrier or within a liquid lipid formulation. The aqueous nanoparticles may be present in volume/volume concentrations between 5% and 50%. One trained in the art can develop similar formulations specific to targeted therapy agents.

Direct Injection into Tumor

In certain embodiments, the therapeutic formulations can be delivered directly inside a solid tumor via guided needle. The echogenic properties of the nano particles will show as a cloud with shadow behind to demonstrate proper placement of the treatment formulation.

Inhaled or Sprayed into Bronchial/Lung Tissue

In certain embodiments, a formulation of drug product can be micronized into solid particles <5 microns that can be sprayed into the terminus of a bronchial tube. The powder will be deposited on the endothelial tissue surface and delivered to cancer cells adjacent or directly present in the bronchial/alveolar space. The particles can also be sprayed on a tumor in a laparoscopic procedure or sprayed to a wound surface post surgical removal of the tumor.

### Intravascular Delivery

Thin liquid formulations containing nanoparticles less than 5 microns can be delivered directly to the vasculature and allowed to circulate in the bloodstream for several days until the particles can carrier are absorbed and the treatment agents delivered systemically to the body. in this embodiment the treatment agent is delivered in a sustained release concentration to ensure a steady delivery of treatment agent for 1-5 days or longer depending on the agent.

### Liposomes

In some applications liposomes can act as the delivery reservoir. The therapeutic agent can be contained within liposomes that are contained within an aqueous carrier hydrogel. Liposomes may be used where the agent can be introduced directly into the tumor cells. Liposomes can also retain aqueously soluble agents where the carrier cannot be a lipid based carrier. The liposomes can be loaded with immune system stimulators such as, but not limited to interleukin-2, other cytokines, and the like. The localized strong immune response could create a response to tumor tissue located next to the implanted hydrogel/liposome mass and act as an effective treatment to the tumor/target tissue.

### Abnormal Tissue Growth Treatment

Antitumor agents can be encapsulated within the liposomes and then used to create a hydrogel/liposome mass that can be delivered to target tissues to kill undesired benign/non-cancerous growths or used to target cancerous tissues. Localized placement of antitumor agents dramatically decreases systemic effects and reduce symptoms from exposure to antitumor agents, but also maintain a medically effect dose of antitumor agents close to the target tissue/tumor. In certain implementations, this approach is used for the treatment of solid tumor treatment, fibroids, prostrate, keratin growths, and the like. A continuous exposure to an eluting antitumor material near the target tissue ensures the cells are not able to repair themselves and survive treatment. It is frequently the case that systemic impact antitumor agent is limiting factor in treating difficult tumors. Maximizing target tissue exposure while minimizing systemic exposure using the instantly disclosed compositions and methods overcomes this difficulty.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

In the examples that follow, Ropivacaine base is used as a proxy for antitumor agents, such as docetaxel, Paclitaxel, Temsirolimus, Rapamycin, and VEGF-TKIs, such as sorafenib, cabozantinib and other non-polar agents with low aqueous solubility. Ropivacaine has a molecular weight of 274 and is considered to be a small molecule API as are most hydrophobic chemotherapeutic agents like Docetaxel with a Mw=807. One skilled in the art can conclude that with non-biologic hydrophobic small molecule agents would have a similar elution behavior to Ropivacaine since there is no chemical or ionic interaction with the excipients. Using ropivacaine as a surrogate is a safer way to conduct elution studies in rats.

### Example 1—Formulation A

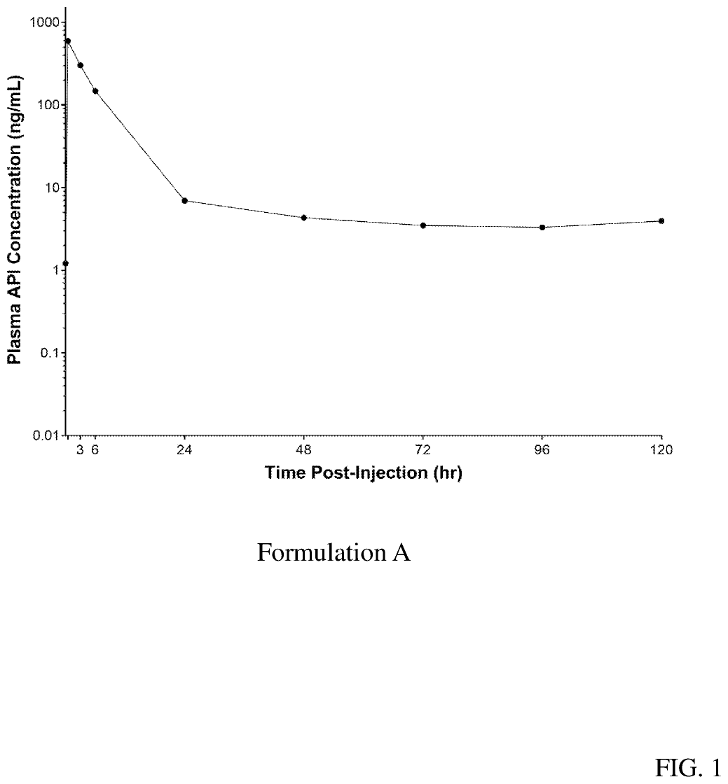

Carnauba Wax/Caprylic acid microparticles (90/10) with carnauba wax/caprylic acid (10/90) secondary lipid phase. The lipid phases contain 130 mg of ropivacaine base per gram of lipid phase. 30% lipid phase total drug product volume. 66/34 MP to secondary lipid phase ratio. Crosslinked hydrogel in aqueous phase. Formulation A contains solid lipid based microparticles containing a surrogate hydrophobic API, ropivacaine, that demonstrates a sustained release profile (as shown in FIG. 1) for at least 120 hours. Formulation A contains solid lipid microparticles within a secondary lipid liquid phase and contained in a continuous aqueous phase.

Rats were injected intramuscularly with 0.1-0.13 mL of drug product near the sciatic nerve and monitored for cardiovascular and CNS adverse events. Blood samples were taken out to 120 hours and ropivacaine concentration in the blood plasma analyzed. As shown in FIG. 1, the formulation demonstrated sustained release behavior out to greater than 120 hours.

### Example 2—Formulation B

Lauric acid/Caprylic acid MPs (90/10) with lauric/caprylic (10/90) secondary lipid phase. 130 mg of ropivacaine in lipid phases. 30% lipid phase by volume. 66/34 microparticle to secondary lipid phase ratio by volume. Formulation B contains solid lipid based microparticles containing a surrogate hydrophobic API, ropivacaine, that demonstrates a sustained release profile for at least 120 hours. Formulation B contains solid lipid microparticles within a secondary lipid liquid phase and contained in a continuous aqueous phase. Formulation B does not demonstrate the same first order release kinetics as formulation A and for a application where a shorter exposure of a chemotherapeutic agent is desired, formulation B would be preferred. For a clinical application were continuous exposure over long periods of time is necessary, formulation A is preferred.

Rats were injected intramuscularly with 0.1-0.13 mL of drug product near the sciatic nerve and monitored for cardiovascular and CNS adverse events. Blood samples were taken out to 120 hours and ropivacaine concentration in the blood plasma analyzed. The formulation demonstrated sustained release behavior out to greater than 120 hours.

This formulation has similar composition to formulation A but used a lauric acid as the main lipid component vs. carnauba wax in formulation A. Both formulations use caprylic acid as the lipid modifier.

Although the disclosure has been described with reference to preferred embodiments, persons skilled in the art will recognize that changes may be made in form and detail without departing from the spirit and scope of the disclosed apparatus, systems and methods.

What is claims is:

1. A composition for treating cancer comprising:
an aqueous carrier, wherein the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%; and
a lipid phase comprising an antitumor agent, the lipid phase dispersed within the aqueous carrier, wherein the lipid phase comprises a plurality of lipid microparticles.

2. The composition of claim 1, wherein a salt form of the antitumor agent unbound by the plurality of lipid microparticles is dissolved in the aqueous carrier.

3. The composition of claim 1, wherein a biologic antitumor agent unbound by the plurality of lipid microparticles is dissolved in the aqueous carrier.

4. The composition of claim 3, wherein the biologic antitumor agent is Bevacizumab.

5. The composition of claim 1, wherein the volumetric ratio between the aqueous carrier and the lipid microparticles is from about 70-80 the aqueous carrier to about 30-20 lipid microparticles.

6. The composition of claim 1, wherein the lipid microparticles comprise one or more fatty acids having an even number of carbons.

7. The composition of claim 6, wherein the one or more fatty acids comprise less than 50% of the total lipid composition of the lipid microparticle.

8. The composition of claim 1, wherein the lipid microparticles comprise one or more fatty acids having an odd number of carbons.

9. The composition of claim 8, wherein the one or more fatty acids are chosen from: stearic acid, oleic acid, myristic acid, caprylic acid, capric acid, lauric acid, palmitic acid, arachidic acid, lignoceric acid, cerotic acid, and mixtures of the forgoing and wherein the melting point of the lipid microparticle is above 37° C.

10. The composition of claim 9, wherein the one or more fatty acids comprise a mixture of steric acid and oleic acid and wherein the ratio of steric acid to oleic acid is about 90:10.

11. The composition of claim 10, wherein in the lipid microparticles comprise about 12% myristic acid, about 32% palmitic acid, about 10% stearic acid, and about 10% oleic acid.

12. The composition of claim 11, wherein the lipid microparticles comprise a mixture of lauric acid and caprylic acid, caproic acid, and/or oleic acid.

13. The composition of claim 5, wherein the lipid microparticle comprises a paraffin, a triglyceride, and/or a wax.

14. The composition of claim 13, wherein the lipid microparticles comprise a mixture of carnauba wax and caprylic acid, caproic acid, and/or oleic acid.

15. The composition of claim 1, wherein the plurality of lipid microparticles comprises a first plurality of lipid microparticles and a second plurality of lipid microparticles and wherein the first plurality of lipid microparticles is solid at about 37° C. and the second plurality of lipid microparticles is liquid at 37° C.

16. The composition of claim 1, wherein the lipid microparticle is not a liposome.

17. The composition of claim 1, wherein the antitumor agent is selected from anthracyclines, mTOR inhibitors, VEGF-TKI agents, and immune stimulators.

18. The composition of claim 1, wherein the antitumor agent is doxorubicin.

19. The composition of claim 1, wherein the plurality of lipid microparticles range from about 5 μm to about 20 μm.

20. The composition of claim 1, wherein the plurality of lipid microparticle are about 5 μm or less.

21. A method of treating cancer in a subject in need thereof comprising administering to the subject and effective amount of a composition comprising:
a hydrogel binding matrix; and a plurality of lipid microparticles dispersed within the hydrogel and comprising one or more antitumor agent.

22. The method of claim 20, wherein the composition is administered directly to the tumor site by guided needle, laparoscopically or post surgically after removal of the tumor.

23. The method of claim 21, wherein the composition is administered by way of injection into a solid tumor by way of a guide needle and wherein tumor targeting is verified via ultrasound imaging.

24. The method of claim 20, wherein the antitumor agent is eluted from the composition over a period of between about 4 and about 7 days.
